(19) United States
(12) Patent Application Publication
(10) Pub. No.: US 2015/0004096 A1
(43) Pub. Date: Jan. 1, 2015

(54) Tumorspecific SPECT/MR(T1), SPECT/MR(T2) and SPECT/CT contrast agents

(71) Applicants: János BORBÉLY, Debrecen (HU); István HAJDU, Tiszacsege (HU); Magdolna BODNÁR, Debrecen (HU); Zsuzsanna CSIKÓS, Nyirbator (HU)

(72) Inventors: János BORBÉLY, Debrecen (HU); István HAJDU, Tiszacsege (HU); Magdolna BODNÁR, Debrecen (HU); Zsuzsanna CSIKÓS, Nyirbator (HU)

(73) Assignee: BBS Nanotechnology Ltd., Debrecen (HU)

(21) Appl. No.: 14/133,975

(22) Filed: Dec. 19, 2013

Related U.S. Application Data
(60) Provisional application No. 61/840,483, filed on Jun. 28, 2013.

(51) Int. Cl. A61K 51/12; A61K 51/06

(52) U.S. Cl. CPC A61K 51/1251; A61K 51/065
USPC 424/1.37

(57) ABSTRACT

The invention relates to cancer receptor-specific bioprobes for single photon emission computed tomography (SPECT) and computed tomography (CT) or magnetic resonance imaging (MRI) for dual modality molecular imaging. The base of the bioprobes is the self-assembled polyelectrolytes, which transport gold nanoparticles as CT contrast agents, or SPION or Gd(III) ions as MR active ligands, and are labeled using complexing agent with technetium-99m as SPECT radiopharmacon. Furthermore these dual modality SPECT/CT and SPECT/MR contrast agents are labeled with targeting moieties to realize the tumorspecificity.

This application claims priority to U.S. provisional application Ser. No. 61/840,483, filed Jun. 28, 2013, the entire disclosure of which is hereby incorporated by reference herein.

### FIELD OF THE INVENTION

The invention relates to cancer receptor-specific bioprobes for single photon emission computed tomography (SPECT) and computed tomography (CT) or magnetic resonance imaging (MRI) for dual modality molecular imaging. The base of the bioprobes is the self-assembled polyelectrolytes, which transport gold nanoparticles as CT contrast agents, or SPION or Gd(III) ions as MR active ligands, and are labeled using complexing agent with technetium-99m as SPECT radiopharmacon. Furthermore these dual modality SPECT/CT and SPECT/MR contrast agents are labeled with targeting moieties to realize the tumorspecificity.

### BACKGROUND OF THE INVENTION

Combining two or more different imaging modalities using multimodal probes can be considerable value in molecular imaging, especially for cancers that are difficult to diagnose and treat. This synergistic combination of imaging modalities, commonly referred to as image fusion, ensures enhanced visualization of biological targets, thereby providing information on all aspects of structure and function, which is difficult to obtain by a single imaging modality alone.

Single photon emission computed tomography, SPECT, allows noninvasive determination of in vivo biodistribution of radiotracers at picomolar concentrations. Using specific radiolabeled probes, obtaining functional information with high sensitivity about molecular processes is possible. SPECT images, however, have limited spatial resolution and lack anatomical details for reference, making the precise localization of lesions difficult. Co-registration of SPECT with anatomical images, from CT or from MR has been commonly used in the clinic to address this problem. The nanomedicine approach uses targeted nanoparticles as platforms to design imaging probes for cancer and other human disorders. In the computed tomography (CT) particular, nanoparticles of gold are suitable for diagnosis of various different types of cancers. On the molecular imaging front, gold with a K-edge at 80.7 keV has higher absorption than iodine (K-edge at 33 keV), thus minimizing bone and tissue interference, which results anatomical references in better contrast with a lower x-ray dose.

### THE STATE OF THE ART

U.S. Pat. No. 7,976,825 relates to macromolecular contrast agents for magnetic resonance imaging.

Biomolecules and their modified derivatives form stable complexes with paramagnetic ions thus increasing the molecular relaxivity of carriers. The synthesis of biomolecular based nanodevices for targeted delivery of MRI contrast agents is described. Nanoparticles have been constructed by self-assembling of chitosan as polycation and poly-gamma glutamic acids (PGA) as polyanion. The nanoparticles are capable of Gd-ion uptake forming a particle with suitable molecular relaxivity. Folic acid is linked to the nanoparticles to produce bioconjugates that can be used for targeted in vitro delivery to a human cancer cell line.

WO06042146 relates to conjugates comprising a nanocarrier, a therapeutic agent or imaging agent and a targeting agent. Disclosed are conjugates comprising a nanocarrier, a therapeutic agent or imaging agent, and a targeting agent, wherein the nanocarrier comprises a nanoparticle, an organic polymer, or both. Compositions comprising such conjugates and methods for using the conjugates to deliver therapeutic and/or imaging agents to cells are also disclosed. The conjugate is a compound having the following formula: A-X-Y wherein A represents the chemotherapeutic agent or imaging agent; X represents the nanoparticle, organic polymer or both, wherein the organic polymer has an average molecular weight of at least about 1,000 daltons; and Y represents the targeting agent.

WO0016811 relates to an MRI contrast agent wherein imaging capability is expressed only within the target abnormal cells, such as tumor, and imaging is not conducted at the site where imaging is not necessary, thereby the detection sensitivity of the abnormal cells such as tumor is improved. Disclosed is an MRI contrast agent, which comprises a complex of a polyanionic gadolinium (Gd) type contrast agent and a cationic polymer, or a complex of a polycationic Gd type contrast agent and an anionic polymer, both complexes being capable of forming a polyion complex, and which expresses an MRI capability at a neutral pH in the presence of a polymer electrolyte.

The state of the art so far failed to provide for the improved compositions according to the present invention.

### SUMMARY OF THE INVENTION

The present invention is directed to novel, targeting dual-modality SPECT/CT and SPECT/MR tumorspecific contrast agents.

For SPECT/CT modality, the fusion nanoparticulate composition comprises (i) at least two polyelectrolyte biopolymers, (ii) targeting molecules conjugated to a polyelectrolyte biopolymer, (iii) gold nanoparticles coated by the polyelectrolyte biopolymer, (iv) optionally a complexing agent conjugated to the polyelectrolyte biopolymer, and (v) a radionuclide, preferably technetium-99m complexed to the nanoparticles.

For SPECT/MR modality, the fusion nanoparticulate composition comprises (i) at least two polyelectrolyte biopolymers, (ii) targeting molecules conjugated to a polyelectrolyte biopolymer, (iii) a complexing agent conjugated to the polyelectrolyte biopolymer, (iv) superparamagnetic iron oxid nanoparticles coated by the polyelectrolyte biopolymer or Gd ions complexed to the polyelectrolyte biopolymer via complexing agents and (v) a radionuclide, preferably technetium-99m complexed to the nanoparticles.

In a preferred embodiment, one of the polyelectrolyte biopolymers is polycation, which is preferably chitosan; and the other of the polyelectrolyte biopolymers is polyanion, which is preferably poly-gamma-glutamic acid.

In a further embodiment, the molecular weight of chitosan in the nanoparticles ranges from about 20 kDa to 600 kDa, and the molecular weight of the poly-gamma-glutamic acid in the nanoparticles ranges from about 50 kDa to 1500 kDa. In a preferred embodiment, the degree of deacetylation of chitosan ranges between 40% and 99%.

For SPECT/CT imaging, the self-assembled nanoparticles comprise gold nanoparticles, which are coated by a polyelectrolyte biopolymer and this system self-assembles with the other biopolymer to produce stable nanosystem for computed tomography.

In a preferred embodiment, the gold nanoparticles are synthesized in situ, in the presence of a polyelectrolyte biopolymer or targeting polyelectrolyte biopolymer. In a preferred embodiment the gold nanoparticles are synthesized in presence of poly-gamma-glutamic acid, or folated poly-gamma-glutamic acid.

For SPECT/MR imaging, nanoparticulate contrast agent contains superparamagnetic iron oxid nanoparticles (SPION) as T2 MR active ligand, or Gd(III) ions as Ti MR active ligands.

In a preferred embodiment, the superparamagnetic iron oxide particles are coated by a polyelectrolyte biopolymer and this system self-assembles with the other biopolymer to produce stable nanosystem for magnetic resonance imaging.

In a further embodiment, the nanoparticles as SPECT/MR fusion contrast agent contain Gd(III) ions as paramagnetic ligands, which are complexed to one of the polyelectrolytes, via the carboxyl groups of polyanion or complexone ligands conjugated to the polycation biopolymer.

In some embodiments, these self-assembled particles internalize into the targeted tumor cells as a consequence of the presence of targeting ligands. The internalized superparamagnetic contrast agents enhance relaxivity, improve the signal-to-noise and therefore conduce to early tumor diagnosis. In a further embodiment, the self-assembled nanosystems contain complexing agents, which can facilitate the radioactively labeling due to the complexing process between the complexing agent and the radiopharmacon. Preferred complexing agents include, but are not limited to: diethylenetriaminepentaaceticacid (DTPA), 1,4,7,10-tetracyclododecane-N,-N′,N″,N′″-tetraaceticacid (DOTA), ethylene-diaminetetraaceticacid (EDTA), 1,4,7,10-tetraazacyclododecane-N,N′,N″-triaceticacid (DO3A), 1,2-diaminocyclohexane-N,N,N′,N′-tetraaceticacid (CHTA), ethyleneglycol-bis(beta-aminoethylether)N,N,N′,N′,-tetraaceticacid (EGTA), 1,4,8,11-tetraazacyclotradecane-N,N′,N″,N′″-tetraaceticacid (TETA), 1,4,7-triazacyclononane-N,N′,N″-triaceticacid (NOTA).

These nanoparticles, as CT or MR contrast agents are radioactively labeled with technetium-99m to produce radiopharmaceutical fusion SPECT/CT or SPECT/MR imaging agent for tumor detection. Targeting moieties are conjugated to one of the self-assembled biopolymers to realize a targeted delivery of imaging agents.

In a preferred embodiment, the targeting agent is preferably folic acid, LHRH, RGD.

In a further embodiment, the nanoparticles have a mean particle size between about 30 and 500 nm, preferably between about 50 and 400 nm, and most preferably between 70 and 250 nm.

The present invention provides fusion imaging agents that are compositions comprising radioactively labeled active nanoparticles. The compositions of the invention target tumor cells, selectively internalize and accumulate in them as a consequence of the presence of targeting ligands, therefore are suitable for early tumor diagnosis.

In its second aspect, the invention relates to a process for the preparation of a targeting contrast composition according to the invention, comprising the steps of

a) contacting of a solution comprising the polyanion, the targeting agent and the MR or CT active ligand, preferably gold nanoparticle with the conjugate of the polycation and the complexing agent; and

b) labeling of the self-assembled nanoparticles.

Furthermore, the invention concerns the use of the targeting contrast composition according to the invention as SPECT/MR or SPECT/CT imaging agents in diagniosis, preferably in cancer diagnosis.

### BRIEF DESCRIPTION OF THE SEVERAL VIEWS OF THE DRAWINGS

FIG. 1 shows the TEM micrograph of poly-γ-glutamic acid coated gold nanoparticles

FIG. 2 shows the size and size distribution of CT active self-assembled nanoparticles.

FIG. 3 represents CT image of CT active self-assembled nanoparticles, Hounsfield unit=70.8 of nanosystem (a) compared with Hounsfield unit=−6.1 of distilled water (b).

FIG. 4 shows the size and size distribution of ^{99m}Tc labeled MRI (T1) active self-assembled nanoparticles.

FIGS. 5A and 5B show the chromatogram of free ^{99m}Tc pertechnetate (FIG. 5A) and ^{99m}Tc labeled nanoparticles (FIG. 5B). Free, unbound ^{99m}Tc was migrated with the solvent to the front line (Rf=1), while the labeled nanoparticle compound was located at the origin (Rf=0). Integrating measured peaks showed the proper ratios of labeled and non-labeled components.

### DETAILED DESCRIPTION OF THE INVENTION

The present invention provides novel, targeting, self-assembled nanoparticles as dual-modality SPECT/MRI or SPECT/CT tumorspecific contrast agent, method for forming them and methods of using these compositions for targeted delivery. The self-assembled particles are provided as nanocarriers, labeled with targeting moieties, containing complexone ligands conjugated to a polycation biopolymer, MR or CT active ligand complexed to the nanoparticles, and a radionuclide complexed to the nanoparticles. These radiolabeled, dual-modality nanoparticles can specifically internalize and accumulate in the targeted tumor cells to realize the receptor mediated uptake. Radiolabeled, targeted nanoparticulate compositions, methods for making these targeting dual-modality contrast agents, radiolabeling and using such compositions in the field of diagnosis and therapy are also provided.

### Nanoparticles, as Contrast Agent Compositions

The present invention is directed to biopolymer-based self-assembled nanocarriers as dual-modality tumorspecific contrast agent for SPECT/MR or SPECT/CT. Biocompatible, biodegradable, polymeric nanoparticles are produced by self-assembly via ion-ion interaction of oppositely charged functional groups of polyelectrolyte biopolymers to form nanocarriers for SPECT and MRI or CT active ligands. In a preferred embodiment, the biopolymers are water-soluble, biocompatible, biodegradable polyelectrolyte biopolymers. One of the polyelectrolyte biopolymers is a polycation, a positively charged polymer, which is preferably chitosan or any of its derivatives. The other of the polyelectrolyte biopolymers is a polyanion, a negatively charged biopolymer. The polyanion is preferably selected from a group consisting of polyacrylic acid (PAA), poly-gamma-glutamic acid (PGA), hyaluronic acid (HA), and alginic acid (ALG).

In a preferred embodiment, the polycation of the nanoparticles ranges in molecular weight from about 20 kDa to 600 kDa, and the polyanion of the nanoparticles ranges in molecular weight from about 50 kDa to 2500 kDa.

In a preferred embodiment, the degree of deacetylation of chitosan ranges between 40% and 99%. The nanoparticles contain targeting moieties necessary for targeted delivery of nanosystems.

The targeting agent is coupled covalently to one of the biopolymers using carbodiimide technique in aqueous media. The water soluble carbodiimide, as coupling agent forms amide bonds between the carboxyl and amino functional groups, therefore the targeting ligand could be covalently bound to one of the polyelectrolyte biopolymers.

In the present invention, the preferred targeting agent is selected from folic acid, lutenizing hormone-releasing hormone (LHRH), and an Arg—Gly—Asp (RGD)-containing homodetic cyclic pentapeptide such as cyclo(-RGDf(NMe)V) and the like.

In a preferred embodiment, the most preferred targeting agent is folic acid, which facilitates the folate mediated uptake of nanoparticles, as tumor specific contrast agents. The nanoparticles of the present invention are preferably targeted to tumor and cancer cells, which overexpress folate receptors on their surface. Due to the binding activity of folic acid ligands, the nanoparticles selectively link to the folate receptors held on the surface of targeted tumor cells, internalize and accumulate in the tumor cells. The folic acid is coupled covalently to the polyanion biopolymer using a carbodiimide technique. The folic acid due to its carboxyl and amino groups can be coupled to the polyanion biopolymer directly or via a PEG-amine spacer.

In a preferred embodiment, the self-assembled nanoparticles are comprised of a polyanion biopolymer, a polycation biopolymer, a targeting agent covalently attached to one of the biopolymers and at least one complexing agent covalently coupled to the polycation.

The complexing agent is coupled covalently to the polycation biopolymer. Water-soluble carbodiimide, as coupling agent is used to make stable amide bonds between the carboxyl and amino functional groups in aqueous media. Using reactive derivatives of complexing agents (e.g. succinimide, thiocyanete), the polycation-complexone conjugate can be directly formed in one-step process without any coupling agents. The nanoparticles can make stable complex with the radionuclide metal ions and for SPECT/MRI T1 modality, paramagnetic ions through these complexone ligans.

In a preferred embodiment, the complexing agents are preferably diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives. More preferably, the complexing agents are DOTA, DTPA, EDTA and NOTA, most preferably DTPA for paramagnetic ligand and NOTA for radionuclide metal ions. The targeted, dual-modality self-assembled nanoparticles described herein are radiolabeled with a radionuclide metal ion, which is preferably Tc-99m as SPECT active ligand.

In a preferred embodiment, the radionuclide metal ions are homogeneously distributed throughout the self-assembled nanoparticle. The radionuclide metal ions can make stable complex with the free complexing agents attached to the polycation biopolymer, therefore they could be performed homogeneously dispersed.

For the formation of the dual-modality SPECT/MR tumorspecific contrast agents, T1 or T2 ligands are conjugated to the nanocarriers, and thereafter radiolabelling with radionuclide technetium (⁹⁹Tc) is carried out.

For T1 MRI modality, paramagnetic ions are complexed to the nanocarriers. The paramagnetic ions are preferably lanthanide or transition metal ions, more preferably gadolinium-, manganese-, chromium-ions, most preferably gadolinium ions, useful as MRI contrast agent.

The paramagnetic ions are homogeneously distributed throughout the self-assembled nanoparticle. The paramagnetic ions can make stable complex with the complexone ligands attached to the polycation biopolymer, therefore they could be performed homogeneously dispersed.

For T2 modality, superparamagnetic ligand, preferably superparamagnetic iron oxide nanoparticles are conjugated to a polyelectrolyte biopolymer, and they are preferably homogenously dispersed. The superparamagnetic iron oxide nanoparticles (SPION) are synthesized in situ in the presence of the polyanion, and then the self-assembling with the polycation is performed.

Size of dried SPIONs ranges between 1 and 15 nm, preferably 3 and 5 nm.

To achieve the dual-modality SPECT/CT tumorspecific contrast agents, gold nanoparticles are conjugated to the nanocarriers, and thereafter radiolabelling with radionuclide technetium is carried out.

The gold nanoparticles are synthesized in situ in the presence of the polyanion, and then the self-assembling with the polycation is performed.

In a preferred embodiment, the nanoparticles described herein have a hydrodynamic diameter between about 30 and 500 nm, preferably between about 50 and 400 nm, and the most preferred range of the hydrodynamic size of nanoparticles is between 70 and 250 nm.

### Methods of Making Nanoparticles, as Dual-Modality Contrast Agent Compositions

The present invention is directed to novel, radiolabeled, biocompatible, biodegradable, targeting nanoparticles as dual-modality SPECT/MRI or SPECT/CT contrast agents. The nanoparticle compositions described herein are prepared by self-assembly of oppositely charged polyelectrolytes via ion-ion interaction between their functional groups. The targeting ligands are conjugated covalently to one of the polyelectrolyte biopolymers and the complexing agents covalently coupled to the polycation biopolymer. These nanoparticles can contain paramagnetic ligand as MRI T1, suparparamagnetic ligands as MRI T2 agents or gold nanoparticles as CT active ligands. These targeted nanoparticles are radioactively labeled with Tc-99m radionuclide to produce dual-modality fusion contrast agents.

In a preferred embodiment, the targeting ligand is attached to one of the biopolymers covalently. The targeting agent is preferably folic acid, LHRH, RGD, the most preferably folic acid.

Folic acid is coupled covalently to the polyanion biopolymer using the carbodiimide technique. Folic acid due to its carboxyl and amino groups can be coupled to the polyanion biopolymer directly or via a PEG-amine spacer.

The polyanion via its reactive carboxyl functional groups can form stable amide bond with the amino functional groups of folic acid or the folic acid-PEG amino spacer using the carbodiimide technique. A folated biopolymer meaning a folated polyanion can be used for the formation of nanoparticles, as targeted dual-modality contrast agent.

In a preferred embodiment, the polycation derivatives namely polycation-complexone polyelectrolyte derivatives are used for the formation of self-assembled nanoparticles. These derivatives of polycation are produced by coupling complexing agent to it covalently. Water soluble carbodiimide is used as coupling agent to form stable amide linkage between the amino groups of polycation and carboxyl groups of complexing agent. Using reactive derivatives of complexing agents (e.g. succinimide, thiocyanete), the polycation-complexone conjugate can be directly formed in one-step process without any coupling agents. In the present invention several complexing agent having reactive carboxyl groups are used to make stable complex with metal ions and therefore afford the possibility to use these systems as imaging agent.

For the formation of a conjugate, the concentration of the biopolymer ranges between about 0.05 mg/ml and 5 mg/ml, preferably 0.1 mg/ml and 2 mg/ml, and the most preferably 0.3 mg/ml and 1 mg/ml.

The overall degree of substitution of complexing agent in the polycation-complexone conjugate is generally in the range of about 1-50%, preferably in the range of about 5-30%, and most preferably in the range of about 10-20%.

Two types of polycation-complexone conjugate can be used for the formation of nanoparticles: (i) a polycation-complexone conjugate, where the complexing agent specific to the radionuclide is covalently attached to the polycation; and (ii) a polycation-complexone conjugate, when two different complexing agents are covalently coupled to the polycation biopolymer, one of them is specific to the paramagnetic ligand and the other is to the radionuclide.

In a preferred embodiment, nanoparticulate compositions, as targeted, dual-modality SPECT/MRI T1 contrast agents are provided. The T1 MR active agent is a paramagnetic ligand, which is preferably a lanthanide or transition metal ion, more preferably a gadolinium-, a manganese-, a chromium-ion, most preferably a gadolinium ion, useful for MRI. The preferred paramagnetic ions can make stable complex with the targeting, self-assembled nanoparticles due to the complexing agents covalently conjugated to polycation.

The gadolinium-chloride solution was used as simple aqueous solution without any pH adjusting. In a preferred embodiment, concentration of gadolinium ion ranges between about 0.2 mg/ml and 1 mg/ml, most preferably between 0.4 mg/ml and 0.5 mg/ml. The molar ratio of said gadolinium ions and complexone conjugated to the polycation ranges preferably between 1:10 and 1:1, more preferably 1:5 and 1:1, and most preferably 1:1.

In a preferred embodiment, nanoparticulate compositions, as targeted, dual-modality SPECT/MRI T2 contrast agents are provided. The T2 MR active agent is a superparamagnetic ligand, preferably iron-oxide ligand, which is preferably nanoparticulate iron-oxide (SPION), which is complexed to a polyelectrolyte biopolymer, and preferably homogenously dispersed.

The superparamagnetic iron oxide nanoparticles are produced in situ in presence of polyanion or targeted polyanion biopolymers, therefore superparamagnetic iron oxide particles are coated by a polyelectrolyte biopolymer.

The SPION synthesis can be performed using several types of Fe(III) and Fe(II) ions, such as pl. FeCl₃xnH₂O (hydrate), Fe₂(SO₄)₃, Fe(NO₃)₃, Fe(III)-phosphate, FeCl₂xnH₂O, FeSO₄xnH₂O (hydrate), Fe(II)-fumarate, or Fe(II)-oxalate.

Preferably, the concentration of polyanion is between 0.01-2.0 mg/ml, the ratio of the Fe(III) and Fe(II) ions ranges between 5:1 and 1:5. The reaction takes place at elevated temperature ranging between 45 and 90° C. under N₂ atmosphere.

In a preferred embodiment, nanoparticulate compositions, as targeted, dual-modality SPECT/CT contrast agents are provided. The CT active ligands are gold nanoparticles with size range of 2-15 nm, preferably 5-12 nm. The gold nanoparticles are produced in situ in presence of polyanion or targeted polyanion biopolymer, therefore gold nanoparticles are homogenously dispersed and coated by the polyelectrolyte biopolymer.

Preferably, the concentration of the polyanion is between 0.01-3.0 mg/ml, the molar ratio of AuCl₃ and polyanion monomers ranges between 2:1 and 5:1. Synthesis of gold nanoparticles in situ in presence of polyanion may be performed using sodium borohydride as reducing agent and optionally sodium citrate dehydrate as stabilizing agent. The molar ratio of gold chloride, sodium borohydride and optionally sodium citrate dehydrate is 1:1:1.

For the production of dual modality contrast agents, the T1 MR, T2 MR or CT active ligand bearing nanoparticles are radioactively labeled with SPECT active radionuclide ligand, which is preferably Tc-99m ion. The preferred radioactive metal ions can make stable complex with the targeting, self-assembled nanoparticles due to the complexing agents, which are covalently conjugated to polycation. In the last step, targeted, self-assembled nanoparticles are radiolabeled with Tc-99m to produce dual modality radiodiagnostic imaging agents. The radiolabeling takes place in physiological salt solution.

For labeling, SnCl₂ (x2H₂O) as reducing agent is added to nanoparticles, then generator-eluted sodium pertechnetate (^{99m}TcO₄⁻) is added to the solvent. The incubation temperature for radiolabeling is room temperature, the incubation time for radiolabeling ranges preferably between 2 min and 120 min, more preferably 5 min and 90 min, and the most preferably 30 min and 60 min.

The nanocarrier formation of the present invention can be obtained in several steps. For the production of a SCECT/MR T1 dual-modality contrast agent, a solution of the targeted polyanion and the polycation-complexone are mixed to form stable, self-assembled nanoparticles, and then an aqueous solution of paramagnetic ions is added to these nanoparticles to make stable paramagnetic nanoparticulate contrast agent. Thereafter these paramagnetic nanoparticles are radioactively labeled with Tc-99m SPECT active radionuclide metal ions to produce the fusion contrast agent.

For production of SPECT/MR T2 dual-modality contrast agent, solution of targeted, SPION-loaded polyanion and polycation-complexone are mixed to form stable, superparamagnetic self-assembled nanoparticles. After that these superparamagnetic nanoparticles are radioactively labeled with Tc-99m SPECT active radionuclide metal ions to produce the fusion contrast agent.

For the production of a SPECT/CT dual-modality contrast agent, a solution of the targeted, gold nanoparticles-loaded polyanion and the polycation-complexone are mixed to form stable, superparamagnetic self-assembled nanoparticles. Then these CT active nanoparticles are radioactively labeled with Tc-99m SPECT active radionuclide metal ions to produce the fusion contrast agent.

The nanoparticle compositions of present invention are prepared by mixing an aqueous solution of the biopolymers at given ratios and order of addition. The polyelectrolytes have statistical distribution inside the nanoparticles to produce globular shape of the nanosystems.

The size of nanoparticles can be controlled by several reaction conditions, such as the concentration of biopolymers, the ratio of biopolymers, and the order of addition. The pH of the biopolymer solution is one of the main factors, which influence the nanoparticle formation due to the surface charge of biopolymers. The charge ratio of biopolymers depends on the pH of the environment. In preferred embodiment, for the nanoparticle formation, the pH of the polycation or its derivatives varies between 3.5 and 6.0, and the pH of the aqueous solution of the polyanion or its derivatives ranges between 7.5 and 9.5.

Biopolymers with high charge density form stable nanoparticles due to these given pH values. The surface charge of nanoparticles could be influenced by several reaction parameters, such as ratio of biopolymers, ratio of residual functional groups of biopolymers, pH of the biopolymers and the environment, etc. The electrophoretic mobility values of nanoparticles, showing their surface charge, could be positive or negative, preferably negative, depending on the reaction conditions described above.

In a preferred embodiment, the concentration of biopolymers ranges between about 0.005 mg/ml and 2 mg/ml, preferably between 0.2 mg/ml and 1 mg/ml, most preferably 0.3 mg/ml and 0.5 mg/ml. The concentration ratio of biopolymers mixed is about 2:1 to 1:2, most preferably about 1:1. The biopolymers are mixed in a weight ratio of 6:1 to 1:6, most preferably 3:1 to 1:3.

### Methods of Using Nanocarrier Compositions

The radiolabeled, targeting dual-modality nanoparticle compositions are useful for targeted delivery of radionuclide metal ions MR or CT active ligands coupled or complexed to the nanoparticles. The present invention is directed to methods of using the above-described nanoparticles, as targeted, dual-modality SPECT/MR or SPECT/CT contrast agents.

The nanoparticles as nanocarriers deliver the imaging agents to the targeted tumor cells in vitro, therefore can be used as targeted, dual-modality SPECT/MR or SPECT/CT contrast agents. The radiolabeled nanoparticles internalize and accumulate in the targeted tumor cells, which overexpress folate receptors, to facilitate the early tumor diagnosis. The side effect of these contrast agents is minimal, because of the receptor mediated uptake of nanoparticles.

In a preferred embodiment, the radioactively labeled, targeted dual-modality imaging agents are stable at pH 7.4, they may be injected intravenously. Based on the blood circulation, the nanoparticles could be transported to the area of interest.

The osmolarity of nanosystems was adjusted using formulating agents. The formulating agent was selected from the group of glucose, physiological salt solution, phosphate buffered saline (PBS), sodium hydrogen carbonate and other infusion base solutions.

The ability of the radiopharmaceutical, dual-modaity nanoparticles to be internalized was studied in cultured cancer cells, which overexpresses folate receptors using confocal microscopy and flow cytometry.

Specific localization, accumulation and biodistribution of these radioactively labeled targeted nanoparticles were investigated in vivo using tumor induced animal. Targeted, radiolabeled nanoparticles specifically internalize into the tumor cells overexpressing folate receptors on their surface. The specific localization was examined by SPECT/MR and SPECT/CT methods, and the biodistribution was estimated by quantitative ROI analysis.

### EXAMPLES

### Example 1

### Preparation of Folated Poly-Gamma-Glutamic Acid (γ-PGA)

Folic acid was conjugated via the amino groups to γ-PGA using carbodiimide technique. γ-PGA (m=60 mg) was dissolved in water (V=100 ml) to produce aqueous solution. The pH of the polymer solution was adjusted to 6.0. After the dropwise addition of cold water-soluble 1-[3-(dimethylamino)propyl]-3-ethylcarbodiimide methiodide (CDI) (m=13 mg in 2 ml distilled water) to the γ-PGA aqueous solution, the reaction mixture was stirred at 4° C. for 1 h, then at room temperature for 1 h. After that, folic acid (m=22 mg in dimethyl sulfoxide, V=10 ml) was added droppwise to the reaction mixture and stirred 4° C. for 1 h, then at room temperature for 24 h. The folated poly-γ-glutamic acid (γ-PGA-FA) was purified by dialysis.

### Example 2

### Preparation of Folated Poly-Gamma-Glutamic Acid

Synthesis of folated PGA was performed in a two steps process. First PEG amine was coupled to FA based on a well-known reaction describe elsewhere. JACS, 130 (2008) 114671 After that FA-PEG amine was conjugated via the amino groups to PGA using carbodiimide technique: γ-PGA (m=300 mg) was dissolved in water (V=300 ml) to produce aqueous solution at a concentration of 1 mg/ml. The pH of the polymer solution was adjusted to 6.0. After addition of 1-hydroxybenzotriazole hydrate (m=94 mg), the reaction mixture was sonicated for 5 min The reaction mixture was cooled to 4° C. and cold water-soluble 1[3-(dimethylamino)propyl]-3-ethylcarbodiimide hydrochloride (EDC) (m=445 mg in V=15 ml water) was added dropwise to the γ-PGA aqueous solution. The reaction mixture was stirred at 4° C. for 10 min, then folic acid-PEG-amine solution (m=100 mg in V=15 ml water) and triethylamine (V=324 μl) were added dropwise to the reaction mixture. The reaction mixture was stirred for 24 h. The folated poly-γ-glutamic acid (γ-PGA-PEG-FA) was purified using mPES MicroKros Filter Module (10 kD).

### Example 3

### Preparation of Folated Poly-Gamma-Glutamic Acid Coated Gold Nanoparticles

Folated PGA was dissolved in distilled water (V=10 ml) to produce a solution with a concentration of c=0.5 mg/ml. After the dropwise addition of solution of gold (III) chloride hydrate (V=500 μl, c=1.7 mg/ml), solution of sodium citrate tribasic dihydrate (V=75 μl, c=10 mg/ml) was added dropwise to the reaction mixture. Then solution of sodium borohydride (V=40 μl, c=1 mg/ml) was added to the reaction. The reaction mixture was stirred at room temperature for 4 h, after that it was purified by dialysis. (FIG. 1)

### Example 4

### Preparation of Folated Poly-Gamma-Glutamic Acid Coated Iron Oxide (PFS)

The pH of the folated PGA solution (c=0.3 mg/ml, V=30 ml) was adjusted to 2.8. After the dropwise addition of FeCl₃x6H₂O solution (c=0.5 mg/ml, V=13.9 ml), the pH of the reaction mixture was raised to 8.5 and after that it was reduced to 6.0. The reaction mixture was stirred for 30 min under N2 atmosphere, and FeCl₂x4H₂O (m=8.9 mg) was added to the reaction mixture. Reaction temperature was raised to 80° C. and the pH was raised by addition of ammonium solution (V=3 ml, c=12.5 m/m %). Reaction time is 15 min.

### Example 5

### Preparation of Chitosan-DTPA Conjugate

Chitosan (m=15 mg) was solubilized in water (V=15 ml); its dissolution was facilitated by dropwise addition of 0.1 M HCl solution. After the dissolution, the pH of chitosan solution was adjusted to 5.0. After the dropwise addition of DTPA aqueous solution (m=11 mg, V=2 ml, pH=3.2), the reaction mixture was stirred at room temperature for 30 min, and at 4° C. for 15 min after that, CDI (m=8 mg, V=2 ml distilled water) was added dropwise to the reaction mixture and stirred at 4° C. for 4 h, then at room temperature for 20 h. The chitosan-DTPA conjugate (CH-DTPA) was purified by dialysis.

### Example 6

### Preparation of Self-Assembled MRI (T1) Active Nanoparticulate Contrast Agent

CH-DTPA solution (c=0.3 mg/ml, V=1 ml, pH=4.0) was added into γ-PGA-FA solution (c=0.3 mg/ml, V=2 ml, pH=9.5) under continuous stirring. An opaque aqueous colloidal system was gained, which remained stable at room temperature for several weeks at physiological pH. To make complex with Gd³⁺, a solution of Gd(III)-chloride (c=0.4 mg/ml, V=400 μl) was added dropwise to the aqueous colloid system containing targeted self-assembled nanoparticles (γ-PGA-FA/CH-DTPA-Gd) and stirred at room temperature for 30 min.

### Example 7

### Preparation of Self-Assembled MRI (T2) Active Nanoparticles

CH-DTPA solution (c=0.3 mg/ml, V=1 ml, pH=4.0) was added into folated poly-gamma-glutamic acid coated iron oxide (PFS) solution (c=0.3 mg/ml, V=3 ml, pH=9.5) under continuous stirring.

### Example 8

### Preparation of Self-Assembled CT Active Nanoparticles

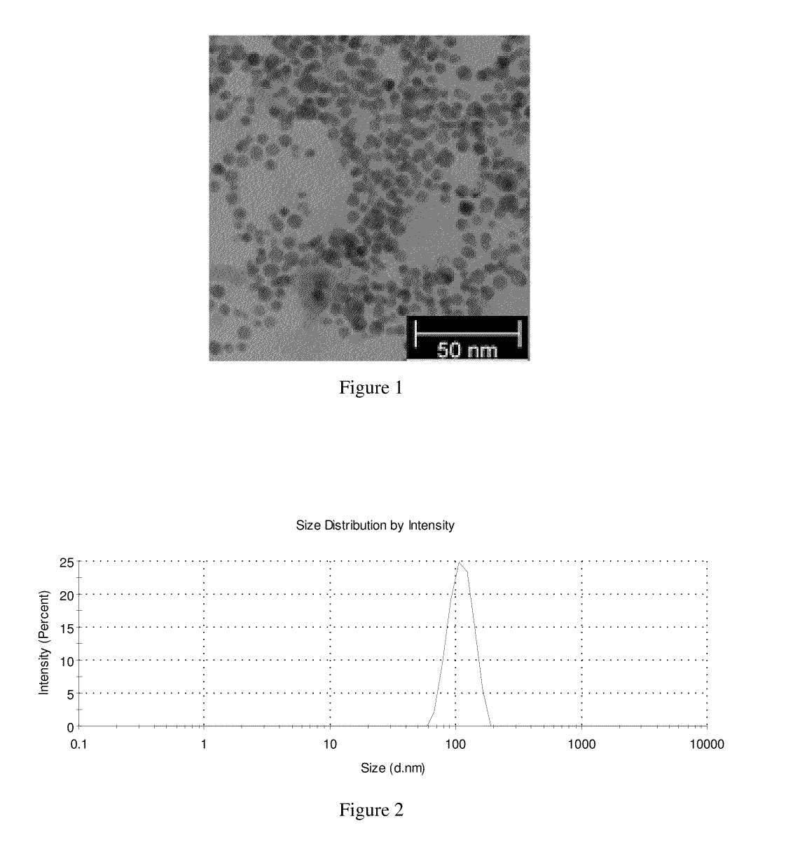

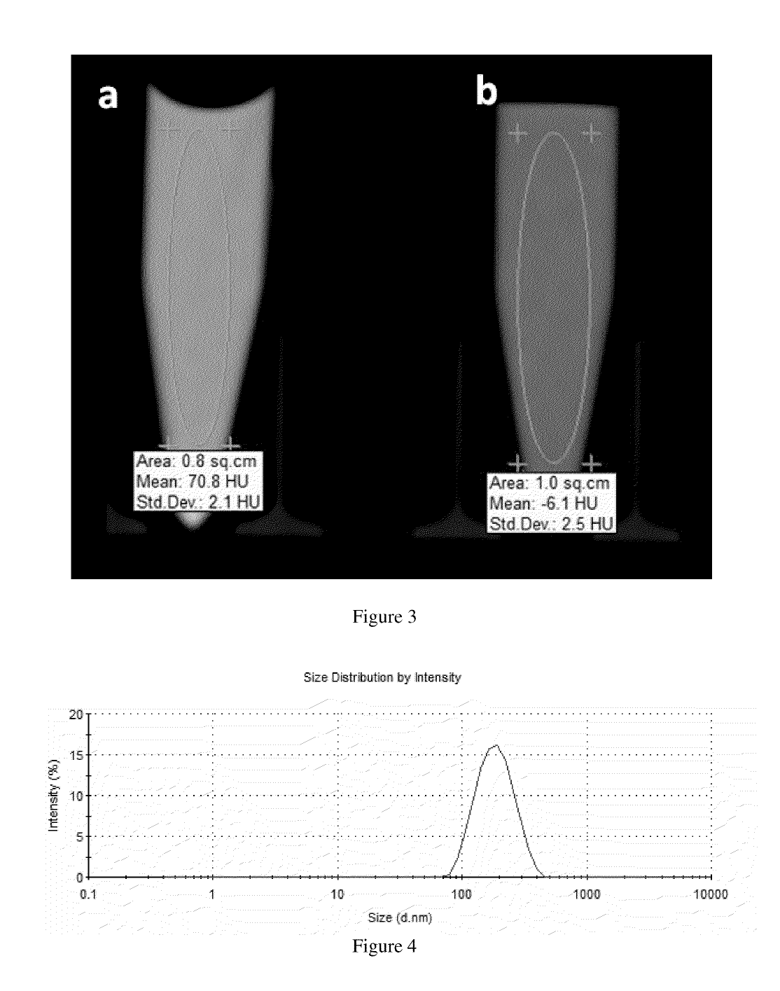

Stable self-assembled nanoparticles were developed via an ionotropic gelation process between the folated poly-γ-glutamic acid coated gold nanoparticles (γ-PGA-FA-gold-NPs), and chitosan-DTPA conjugate (CH-DTPA). Briefly, CH-DTPA solution (c=0.2 mg/ml, V=1 ml, pH=4.0) was added into γ-PGA-FA-gold-NPs solution (c=0.2 mg/ml, V=3 ml, pH=9.5) under continuous stirring. An aqueous colloidal system was gained, which remained stable at room temperature for several weeks at physiological pH. (FIG. 2, 3)

### Example 9

### Labeling Method of Self-Assembled Nanoparticles

For labelling, 40 μg SnCl₂ (x2H₂O) (in 10 μl 0.1 M HCl) as reducing agent was added to 2.6 ml of nanoparticle suspension, then 1 ml (900 MBq activity) of sterile generator-eluted pertechnetate (^{99m}TcO₄—) solution was added to the solvent. Labelling was performed during 60-minute incubation at room temperature. (FIG. 4)

### Example 10

### Characterisation of ^{99m}Tc labeled self-assembled nanoparticles

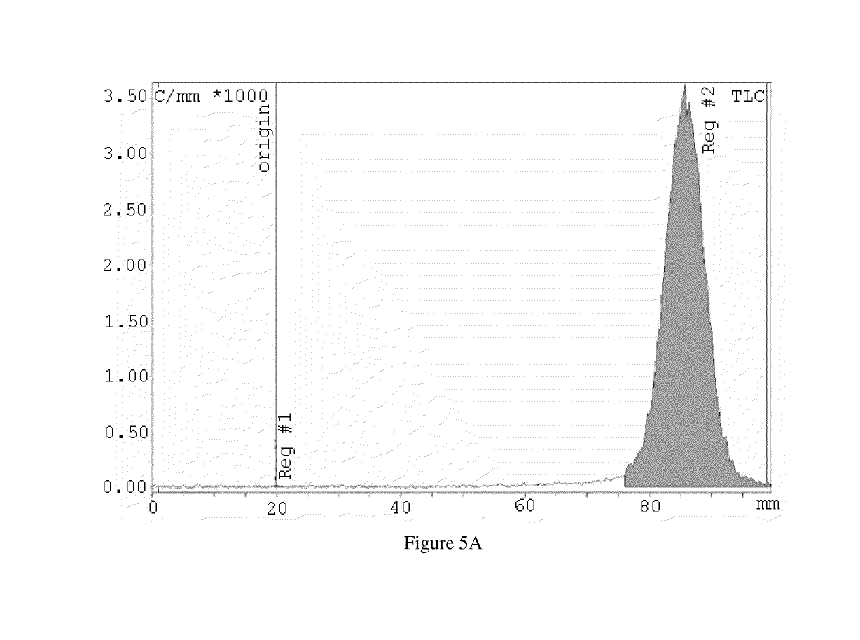

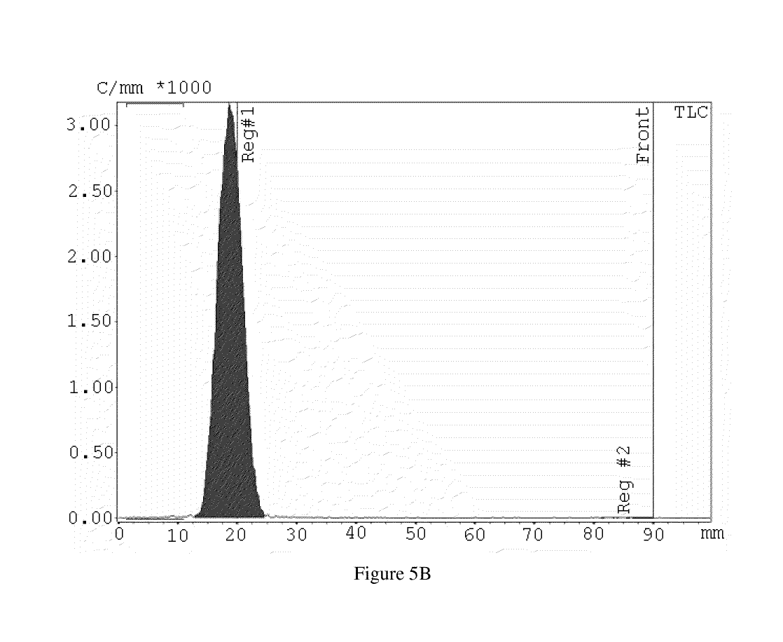

Radiochemical purity was examined by means of thin-layer chromatography, using silica gel as the coating substance on a glass-fibre sheet (ITLC-SG). Plates were developed in methyl ethyl ketone. Free, unbound ^{99m}Tc-pertechnetate migrated with the solvent to the front line, while the labelled nanoparticle compound was located at the origin (bottom). The Raytest MiniGita device (Mini Gamma Isotope Thin Layer Analyzer) was applied to determine the distribution of radioactivity in the developed ITLC-SG plates. The labelling efficiency was examined 1 h, 6 h and 24 h after labeling. Radiochemical samples were stored at room temperature in a dark place. The radiolabeled products showed high degree and durable labelling efficiency above 99% (FIG. 5).

1. A targeting SPECT/CT nanoparticulate tumorspecific contrast composition comprising (i) at least two, preferably water-soluble, biocompatible and biodegradable nanoparticle polyelectrolyte biopolymers; (ii) a targeting molecule conjugated a polyanion biopolymer; (iii) gold nanoparticles coated by the polyelectrolyte biopolymer, (iv) optionally a complexing agent conjugated to the polyelectrolyte biopolymer, and (v) a radionuclide, preferably technetium-99m complexed to the nanoparticles.

2. The targeting SPECT/CT nanoparticulate tumorspecific contrast composition as claimed in claim 1, wherein the self-assembled nanoparticles comprise gold nanoparticles, which are coated by a polyelectrolyte biopolymer.

3. The targeting SPECT/CT nanoparticulate tumorspecific contrast composition as claimed in claim 1, wherein the gold nanoparticles are synthesized in situ, in the presence of a polyelectrolyte biopolymer or a targeting polyelectrolyte biopolymer, preferably in presence of poly-gamma-glutamic acid, or folated poly-gamma-glutamic acid.

4. A targeting SPECT/MR nanoparticulate tumorspecific contrast composition comprising (i) at least two, preferably water-soluble, biocompatible and biodegradable nanoparticle polyelectrolyte biopolymers; (ii) a targeting molecule conjugated a polyanion biopolymer; (iii) a complexing agent conjugated to the polyelectrolyte biopolymer, (iv) lanthanide or transition metal ions, more preferably gadolinium-, manganese-, chromium-ions, most preferably gadolinium ions (as MR T1 contrast agent) complexed to a polyelectrolyte biopolymer via complexing agents, or superparamagnetic iron oxide nanoparticles (as MR T2 contrast agent), said contrast agents preferably coated by a polyelectrolyte biopolymer and (v) a radionuclide, preferably technetium-99m complexed to the nanoparticles.

5. The targeting SPECT/MR nanoparticulate tumorspecific contrast composition as claimed in claim 4, which contains superparamagnetic iron oxide particles as T2 MR active ligand, wherein the superparamagnetic iron oxide particles preferably are coated by a polyelectrolyte biopolymer; or contains Gd(III) ions as T1 MR active ligand

6. The targeting contrast composition as claimed in claim 1, wherein one of the nanoparticle polyelectrolyte biopolymers is a polycation or a derivative thereof, preferably chitosan, and the other one is a polyanion biopolymer or a derivative thereof, preferably selected from the group consisting of polyacrylic acid (PAA), poly-gamma-glutamic acid (PGA) hyaluronic acid (HA), and alginic acid (ALG), preferably poly-gamma-glutamic acid (PGA), said biopolymers being preferably self-assembled based on the ion-ion interactions between their functional groups.

7. The targeting SPECT/MR nanoparticulate tumorspecific contrast composition as claimed in claim 4, wherein the Gd(III) ions are complexed to one of the polyelectrolytes, via the carboxyl groups of the polyanion or complexone ligands conjugated to the polycation biopolymer.

8. The targeting contrast composition as claimed in claim 1, wherein
a) the polycation, preferably the chitosan, has a molecular weight from about 20 and 600 kDa, and the degree of its deacetylation ranges between 40% and 99%;
b) the polyanion, preferably the poly-gamma-glutamic acid (PGA) has a molecular weight between 50 kDa and 1500 kDa; and/or
c) the targeting agent is selected from the group of folic acid, LHRH and an Arg—Gly—Asp (RGD)-containing homodetic cyclic pentapeptide, preferably cyclo(-RGDf(NMe)V), most preferably folic acid, and preferably is conjugated to the polyanion.

9. The targeting contrast composition as claimed in claim 1, wherein the complexing agent is selected from the group consisting of diethylenetriaminepentaacetic acid (DTPA), 1,4,7,10-tetracyclododecane-N,-N′,N″,N′″-tetraaceticacid (DOTA), ethylene-diaminetetraaceticacid (EDTA), 1,4,7,10-tetraazacyclododecane-N,N′,N″-triaceticacid (DO3A), 1,2-diaminocyclohexane-N,N,N′,N′-tetraaceticacid (CHTA), ethyleneglycol-bis(beta-aminoethylether)N,N,N′,N′,-tetraaceticacid (EGTA), 1,4,8,11-tetraazacyclotradecane-N,N′,N″,N″-tetraaceticacid (TETA), 1,4,7-triazacyclononane-N,N′,N″-triaceticacid (NOTA), preferably diethylenetriaminepentaacetic acid (DTPA).

10. The targeting contrast composition as claimed in claim 1, wherein the nanoparticles have a mean particle size between about 30 and 500 nm, preferably between about 50 and 400 nm, and most preferably between 70 and 250 nm.

11. A process for the preparation of a targeting contrast composition as claimed in claim 1, comprising the steps of
a) contacting of a solution comprising the polyanion, the targeting agent and the MR or CT active ligand, preferably the CT active ligand gold nanoparticle with the conjugate of the polycation and the complexing agent; and
b) labeling of the self-assembled nanoparticles by a radionuclide.

12. The process as claimed in claim 11, wherein
a) a polycation-complexone conjugate is used, where the complexing agent specific to the radionuclide is covalently attached to the polycation; or
b) a polycation-complexone conjugate is used, where two different complexing agents are covalently coupled to the polycation biopolymer, one of them is specific to the MR active paramagnetic ligand and the other is to the radionuclide.

13. The process as claimed in claims 11, wherein
a) the concentration of the biopolymer ranges between about 0.05 mg/ml and 5 mg/ml, preferably 0.1 mg/ml and 2 mg/ml, and the most preferably 0.3 mg/ml and 1 mg/ml; and/or
b) the overall degree of substitution of complexing agent in the polycation-complexone conjugate is in the range of about 1-50%, preferably in the range of about 5-30%, and most preferably in the range of about 10-20%; and/or
c) the concentration of gadolinium ion used ranges between about 0.2 mg/ml and 1 mg/ml, most preferably between 0.4 mg/ml and 0.5 mg/ml; and/or
d) the molar ratio of the gadolinium ions and complexone conjugated to the polycation ranges preferably between 1:10 and 1:1, more preferably 1:5 and 1:1, and most preferably 1:1; and/or
e) the gold nanoparticles used are in the size range of 2-15 nm, preferably 5-12 nm;
f) the pH of the polycation or its derivatives varies between 3.5 and 6.0, and the pH of the aqueous solution of the polyanion or its derivatives ranges between 7.5 and 9.5.

14. The process for the preparation of a SPION containing targeting contrast composition as claimed in claim 11, wherein
a) the concentration of the polyanion is between 0.01-2.0 mg/ml, the ratio of the MR active Fe(III) and Fe(II) ions ranges between 5:1 and 1:5; and/or
b) the reaction takes place at elevated temperature ranging between 45 and 90° C. under N₂ atmosphere.

15. The process as claimed in claims 11, wherein the radiolabeling with Tc-99m takes place in physiological salt solution, using SnCl₂ (x2H₂O) as reducing agent, which is added to the nanoparticles, then generator-eluted sodium pertechnetate (^{99m}TcO₄⁻) is added to the solvent at room temperature as incubation temperature, for the time period of preferably between 2 min and 120 min, more preferably 5 min and 90 min, and the most preferably 30 min and 60 min.

16. The process as claimed in claim 11, wherein the preparation takes place in several steps.

17. A method of diagnosis, said method comprising using the targeting contrast composition as claimed in claim 1 as a SPECT/MR or SPECT/CT imaging agent.

18. The method as claimed in claim 16, wherein the targeting contrast composition is used in cancer diagnosis.
